(19) United States
(12) Patent Application Publication
(10) Pub. No.: US 2021/0077065 A1
(43) Pub. Date: Mar. 18, 2021

(54) METHOD AND APPARATUS FOR SIMULTANEOUS 4D ULTRAFAST BLOOD FLOW AND TISSUE DOPPLER IMAGING OF THE HEART AND RETRIEVING QUANTIFICATION PARAMETERS

(71) Applicants: INSERM (INSTITUT NATIONAL DE LA SANTÉ ET DE LA RECHERCHE MÉDICALE), Paris (FR); ECOLE SUPÉRIEURE DE PHYSIQUE ET DE CHIMIE INDUSTRIELLES DE LA VILLE DE PARIS, Paris (FR); CENTRE NATIONAL DE LA RECHERCHE SCIENTIFIQUE - CNRS, PARIS (FR); SORBONNE UNIVERSITE, Paris (FR); UNIVERSITÉ DE PARIS, Paris (FR)

(72) Inventors: Mathieu PERNOT, Paris (FR); Clément PAPADACCI, Paris (FR); Mickael TANTER, Paris (FR)

(21) Appl. No.: 16/970,810

(22) PCT Filed: Feb. 18, 2019
(86) PCT No.: PCT/EP2019/053920

§ 371 Date: Aug. 18, 2020

(30) Foreign Application Priority Data
Feb. 19, 2018 (EP) 18305173.9

(51) Int. Cl. A61B 8/08; A61B 8/06; A61B 8/14; A61B 8/00; G06T 13/20; G06T 7/00; G06T 7/70; G16H 30/40; G16H 30/20

(52) U.S. Cl. CPC A61B 8/0883; G06T 2207/30048; A61B 8/145; A61B 8/4444; A61B 8/5207; A61B 8/5223; A61B 8/54; G06T 13/20; G06T 7/0012; G06T 7/70; G16H 30/40; G16H 30/20; A61B 8/483; A61B 8/488; A61B 8/4494; G06T 2207/10016; G06T 2207/30104; G06T 2207/10136; A61B 8/065

(57) ABSTRACT

The invention relates to the field of ultrasound imaging of the heart. 4D ultrafast ultrasound imaging of the heart is performed and may be used to compute major cardiac echo-graphic Flow and Tissue Doppler index indexes such as E/E′, E/Apex A, E′/A′ with a single acquisition in a very quick time (e.g. with-in a heart beat) and in a reproducible way, independently of the experience of the operator.

### FIELD

The disclosure relates to methods and apparatus for for simultaneous 4D ultrafast blood flow and tissue Doppler imaging of the heart and retrieving quantification parameters.

### BACKGROUND

Echography ultrasound imaging is a portable, fast and low-cost technology that is routinely used in cardiology due to its ability to perform real-time imaging of the heart. Morphological parameters like cavity volumes and dynamic functional detection such as left ventricle outflow tract can be measured for diagnosis in two dimensions (2D) and one dimension (1D) respectively. Many more indexes used to characterize the state of the heart are measured routinely in real-time in one or two dimensions. The choice between 1D and 2D imaging is motivated by the frame-rate needed to measure physiological phenomena. To measure the cavity volume for instance, 2D imaging is more suitable as the frame rate needed to capture the global motion of the heart does not exceed real-time. However, to quantify fast physiological phenomena such as small tissue motion (E′/A′ factor), blood flow speed (E/A factor) or both in the same time (E/E′), 1D imaging is performed to decrease the number of transmitted ultrasonic waves to allow a frame rate increase. Such routine exams take a noticeable amount of time due to manual selection of the region of interest. Moreover, such manual selections induce operator variability.

One advanced type of ultrasound imaging, ultrafast ultrasound imaging, has been largely studied [M. Tanter and M. Fink, “Ultrafast imaging in biomedical ultrasound,” IEEE Trans. Ultrason. Ferroelectr. Freq. Control, in press, January 2014]. It enables to increase the frame rate to reach few kilo images per second. The method relies on the emission of unfocused wave to insonifiate all the medium in few transmits. Recently, ultrafast imaging was extended to 4D ultrasound imaging, i.e. animated 3D ultrasound imaging. In particular, 4D ultrasound ultrafast imaging was performed to image blood flow in the left ventricle of human heart during a cardiac cycle, as well as blood flow and tissue motion of the carotid during a cardiac cycle [J. Provost et al., “3D ultrafast ultrasound imaging in vivo,” Phys. Med. Biol., vol. 59, no. 19, p. L1, October 2014.]

### SUMMARY

Embodiments described therein provide for enhanced methods and apparatus for ultrasound imaging of the heart.

To this end, a method is provided for 4D imaging of a heart of a living being, said method including at least the following steps:
(a) an acquisition step wherein unfocused ultrasonic waves are transmitted in the heart by a 2D array ultrasonic probe and raw data from backscattered ultrasonic waves are acquired by said 2D array ultrasonic probe;
(b) an imaging step wherein a sequence of 3D images is generated from said raw data, said sequence of 3D images forming an animation showing movements of an imaged volume including at least part of the heart;
(c) a velocity computing step wherein 3D cartography of at least one parameter related to blood velocity and tissue velocity are automatically computed in said imaged volume, based on said sequence of 3D images;
(d) a locating step wherein at least one point of interest having a predetermined property is automatically located in said sequence of 3D images;
(e) a quantification step wherein at least one velocity chosen between blood velocity and tissue velocity is automatically determined at said at least one point of interest and a predetermined quantification parameter is automatically computed, involving said at least one velocity,
   wherein the quantification parameter involves:
either a peak blood velocity in a certain anatomic area and said locating step (d) includes automatically locating said point of interest (13) as a point of maximum blood velocity in said anatomic area and in at least part of the sequence of 3D images;
or a peak tissue velocity in a certain anatomic area and said locating step (d) includes automatically locating said anatomic area in the sequence of 3D images and automatically locating said point of interest as a point of maximum tissue velocity in said anatomic area in the sequence of 3D images.

Thanks to these dispositions, the quantification parameter is computed instantly, accurately in a repetitive manner, without any variability due the experience of the operator.

The method may further include one and/or other of the following features:
during said acquisition step (a), said unfocused ultrasonic waves are transmitted in several series of successive unfocused ultrasonic waves, the successive unfocused ultrasonic waves of each series having respectively different propagation directions, and said imaging step (b) includes synthesizing a 3D image by ultrasound synthetic imaging from the respective raw data corresponding to said successive unfocused ultrasonic waves of each series;
each series of successive unfocused ultrasonic waves includes 1 to 100 successive unfocused ultrasonic waves, for instance 1 to 20 successive unfocussed ultrasonic waves;
said unfocused ultrasonic waves transmitted during said acquisition step (a) are divergent;
during said acquisition step (a), said divergent ultrasonic waves have virtual sources behind the 2D array probe (i.e. opposite the direction of transmission of the waves);
during said acquisition step, said unfocused ultrasonic waves are transmitted at a rate of more than 10 000 unfocused ultrasonic waves per second;
the quantification parameter involves a peak blood velocity in a certain anatomic area and said locating step (d) includes automatically locating said anatomic area in the sequence of 3D images and automatically locating said point of interest as a point of maximum blood velocity in the sequence of 3D images;
the quantification parameter involves a temporal variation such as acceleration time or deceleration time or time integral of the blood velocity in a certain anatomic area and said locating step (d) includes automatically locating said anatomic area in the sequence of 3D images and automatically locating said point of interest as a point of maximum blood velocity in the sequence of 3D images;
the quantification parameter involves a blood flow rate or Cardiac Output (CO) obtained by the time and space integral of the blood velocity in a certain anatomic area (e.g. total blood flow-rate entering or leaving the heart according to the time in the cardiac cycle) and said locating step (d) includes automatically locating said anatomic area in the sequence of 3D images and automatically locating said point of interest as a point of maximum blood velocity in the sequence of 3D images;
the quantification parameter involves a peak tissue velocity in a certain anatomic area and said locating step (d) includes automatically locating said anatomic area in the sequence of 3D images and automatically locating said point of interest as a point of maximum tissue velocity in said anatomic area in the sequence of 3D images;
the quantification parameter involves a tissue velocity at a certain anatomic position in the heart and said locating step (d) includes automatically locating said anatomic position in the sequence of 3D images;
the quantification parameter determined at said quantification step is chosen from E, A, E′, A′, S, D, Vp, S′, E/A, E/E′, E/E′, E′/A′, S, S/D, Q, Qsystolic, Qdiastolic, DT, IVRT, PVAT, VTI, Gmean and Gmax wherein:
   E is the early diastolic trans-mitral flow velocity;
   E′ is the early diastolic mitral annular velocity;
   A is late diastolic trans-mitral flow velocity;
   A′ is the late diastolic mitral annular velocity;
   S is the peak pulmonary venous systolic velocity;
   D is the peak pulmonary venous early diastolic velocity;
   Vp is the velocity of flow progression;
   Q is the flow rate or Cardiac Output; Qsystolic is the total output transaortic flow rate and Qdiastolic is the total input transmitral flow rate;
   DT is the e-wave deceleration time;
   PVAT is the pulmonary acceleration time;
   IVRT is the length of the isovolumetric relaxation time;
   Gmean and Gmax are the mean and maximum transvalvular pressure gradients;
   VTI is velocity time integral;
   S′ is the peak systolic annular velocity;
during said acquisition step, said unfocussed ultrasonic waves are transmitted at a rate of more than 10 000 unfocussed ultrasonic waves per second;
during said acquisition step (a), said unfocussed ultrasound waves are transmitted in the heart for a duration of at least part of a cardiac cycle and less than 10 cardiac cycles, for instance less than 5 cardiac cycles;
during said acquisition step (a), said unfocussed ultrasound waves are transmitted in the heart for a duration comprised between 1s and 10s, for instance between 1s and 5s;
at step (d), said at least one point of interest is automatically located based solely on said 3D cartography and its temporal profile, and at step (e), said at least one velocity is automatically determined at said at least one point of interest based solely on said 3D cartography and its temporal profile.

Besides, the disclosure proposes an apparatus for 4D imaging of a heart of a living being, said apparatus including at least a 2D array ultrasonic probe and a control system configured to:
(a) transmit unfocused ultrasonic waves in the heart through said 2D array ultrasonic probe and acquire raw data from backscattered ultrasonic waves through said 2D array ultrasonic probe,
(b) generate a sequence of 3D images from said raw data, said sequence of 3D images forming an animation showing movements of an imaged volume including at least part of the heart,
(c) automatically compute 3D cartography of at least one parameter related to blood velocity and tissue velocity in said imaged volume, based on said sequence of 3D images,
(d) automatically locate at least one point of interest having a predetermined property in said sequence of 3D images,
(e) automatically determine at least one velocity chosen between blood velocity and tissue velocity at said at least one point of interest and automatically compute a predetermined quantification parameter involving said at least one velocity,
   wherein the quantification parameter involves:
either a peak blood velocity in a certain anatomic area and said control system (3, 4) configured to automatically locate said point of interest (13) as a point of maximum blood velocity in said anatomic area and in at least part of the sequence of 3D images;
or a peak tissue velocity in a certain anatomic area and said control system (3, 4) configured to automatically locate said anatomic area in the sequence of 3D images and automatically locating said point of interest as a point of maximum tissue velocity in said anatomic area in the sequence of 3D images.

The apparatus may further include one and/or other of the following features:
the quantification parameter involves a peak blood velocity in a certain anatomic area and said control system is configured to automatically locate said anatomic area in the sequence of 3D images and to automatically locate said point of interest as a point of maximum blood velocity in the sequence of 3D images;
the quantification parameter involves a peak tissue velocity in a certain anatomic area and said control system (is configured to automatically locate said anatomic area in the sequence of 3D images and to automatically locate said point of interest as a point of maximum tissue velocity in said anatomic area in the sequence of 3D images;
the quantification parameter involves a temporal variation such as acceleration time or deceleration time or time integral of the blood velocity in a certain anatomic area and said locating step (d) includes automatically locating said anatomic area in the sequence of 3D images and automatically locating said point of interest as a point of maximum blood velocity in the sequence of 3D images;
the quantification parameter involves a blood flow rate obtained by the time and space integral of the blood velocity in a certain anatomic area (e.g. total blood flow-rate entering or leaving the heart according to the time in the cardiac cycle) and said locating step (d) includes automatically locating said anatomic area in the sequence of 3D images and automatically locating said point of interest as a point of maximum blood velocity in the sequence of 3D images;
the quantification parameter involves a tissue velocity at a certain anatomic position in the heart and said control system is configured to automatically locate said point of interest at said anatomic position in the sequence of 3D images;
the quantification parameter is chosen from E, A, E′, A′, S, D, Vp, S′, E/A, E/E′, E/E′, E′/A′, S, S/D, Q, Qsystolic, Qdiastolic, DT, IVRT, PVAT, VTI, Gmean and Gmax wherein:
   E is the early diastolic trans-mitral flow velocity;
   E′ is the early diastolic mitral annular velocity;
   A is late diastolic trans-mitral flow velocity;
   A′ is the late diastolic mitral annular velocity;
   S is the peak pulmonary venous systolic velocity;
   D is the peak pulmonary venous early diastolic velocity;
   Vp is the velocity of flow progression;
   Q is the flow rate or cardiac output;
   Qsystolic is the total output transaortic flow rate;
   Qdiastolic is the total input transmitral flow rate;
   DT is the e-wave deceleration time;
   PVAT is the pulmonary acceleration time;
   IVRT is the length of the isovolumetric relaxation time;
   Gmean and Gmax are the mean and maximum transvalvular pressure gradients;
   VTI is velocity time integral;
   S′ is the peak systolic annular velocity;
said control system is configured to transmit said unfocussed ultrasonic waves in several series of successive unfocussed ultrasonic waves, the successive unfocussed ultrasonic waves of each series having respectively different propagation directions, and said control system is configured to synthesize a 3D image by ultrasound synthetic imaging from the respective raw data corresponding to said successive unfocussed ultrasonic waves of each series;
each series of successive unfocused ultrasonic waves includes 1 to 100 successive unfocused ultrasonic waves, for instance 1 to 20 successive unfocussed ultrasonic waves;
said control system (3, 4) is configured to transmit said unfocused ultrasonic waves as divergent ultrasonic waves;
said divergent ultrasonic waves have virtual sources behind the 2D array probe (i.e. opposite the direction of transmission of the waves);
said control system is configured to transmit said unfocussed ultrasonic waves at a rate of more than 10 000 unfocussed ultrasonic waves per second;
said control system is configured to transmit said unfocussed ultrasound waves for a duration of at least part of a cardiac cycle and less than 10 cardiac cycles, for instance less than 5 cardiac cycles;
said control system is configured to transmit said unfocused ultrasound waves for a duration comprised between 1 s and 10 s, for instance between 1 s and 5 s;
said control system is configured to:
(d) automatically locate said at least one point of interest based solely on said 3D cartography and its temporal profile;
(e) automatically determine said at least one velocity at said at least one point of interest based solely on said 3D cartography and its temporal profile.

### BRIEF DESCRIPTION OF THE DRAWINGS

Other features and advantages of the disclosure appear from the following detailed description of one non-limiting example thereof, with reference to the accompanying drawings.

In the drawings:

FIG. 1 is a schematic drawing showing an apparatus for 4D imaging of the heart;

FIG. 2 is a block diagram showing part of the apparatus of FIG. 1;

FIG. 3 is a diagram illustrating virtual sources of divergent ultrasound waves, generated by the apparatus of FIGS. 1-2;

FIG. 4 illustrates the transmission of a divergent ultrasound wave in the heart of a living being by the apparatus of FIGS. 1-2;

FIG. 5 illustrates the transmission of two successive divergent ultrasound waves with different directions of propagation, respectively from two virtual sources;

FIG. 6 illustrates results obtained simultaneous 4D ultrafast blood flow and 4D tissue velocity of the left ventricle of a human sound volunteer in a single heartbeat:

FIG. 6(a) shows 5 cross sections of the left ventricle extracted from the sequence of 3D images generated by the apparatus, respectively allowing visualization of the cardiac phases (rapid inflow, diastasis, atrial systole, pre ejection, ejection) which are separated by dotted lines on FIGS. 6(a)-6(d);

FIG. 6(b) is a Doppler spectrogram of blood flow at the mitral valve;

FIG. 6(c) is a tissue velocity curve at the basal septum location; and

FIG. 6(d) is a corresponding electrocardiogram (ECG);

FIG. 7 illustrates images and measurements of the left ventricle, made by a trained operator with a classical 2D clinical ultrasound system for the same volunteer as that of FIG. 6, with indication of cardiac phases as in FIG. 6:

FIG. 7(a) shows the Doppler spectrum of blood flow at the mitral valve and

FIG. 7(b) shows tissue velocity at the basal septum obtained with a clinical ultrasound system for the healthy volunteer; and

FIGS. 8 and 9 are respectively similar to FIGS. 6-7, for a patient with a hypertrophic cardiomyopathy.

### MORE DETAILED DESCRIPTION

In the Figures, the same references denote identical or similar elements.

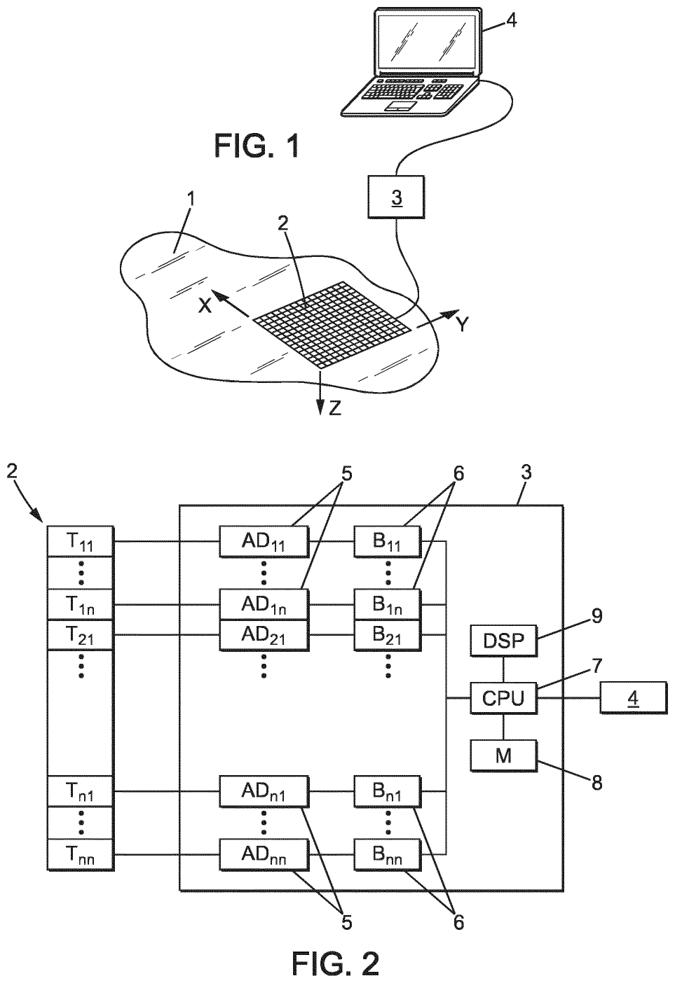

The apparatus shown on FIGS. 1 and 2 is adapted to ultrafast 4D ultrasound imaging of the heart of a living being 1, for instance a mammal and in particular a human. The apparatus may include for instance at least a 2D array ultrasonic probe 2 and a control system.

The 2D array ultrasonic probe 2 may have for instance a few hundreds to a few thousands transducer elements Tᵢⱼ, with a pitch lower than 1 mm. The 2D array ultrasonic probe 2 may have n*n transducer elements disposed as a matrix along two perpendicular axes X, Y, transmitting ultrasound waves along an axis Z which is perpendicular to the XY plane. In one specific example, the 2D array ultrasonic probe 2 may have 1024 transducer elements Tᵢⱼ (32*32), with a 0.3 mm pitch. The transducer elements may transmit for instance at a central frequency comprised between 1 and 10 MHz, for instance of 3 MHz.

The control system may for instance include a specific control unit 3 and a computer 4. In this example, the control unit 3 is used for controlling 2D array ultrasonic probe 2 and acquiring signals therefrom, while the computer 4 is used for controlling the control unit 3, generating 3D image sequences from the signals acquired by control unit 3 and determining quantification parameters therefrom. In a variant, a single electronic device could fulfill all the functionalities of control unit 3 and computer 4.

As shown on FIG. 2, control unit 3 may include for instance:
n*n analog/digital converters 5 (ADᵢⱼ) individually connected to the n transducers Tᵢⱼ of 2D array ultrasonic probe 2;
n*n buffer memories 6 (Bᵢⱼ) respectively connected to the n*n analog/digital converters 5;
a central processing unit 7 (CPU) communicating with the buffer memories 6 and the computer 4;
a memory 8 (MEM) connected to the central processing unit 7;
a digital signal processor 9 (DSP) connected to the central processing unit 7.

The apparatus may operate as follows.

(a) Acquisition:

The 2D array ultrasonic probe 2 is placed on the chest 10 of the patient 1, usually between two ribs, in front of the heart 12 of the patient as shown in FIG. 4. Because of the limited intercostal space between ribs 11 compared to the size of the heart 12 to be imaged, the 2D array ultrasonic probe 2 is controlled to transmit divergent ultrasonic waves in the chest 10, for instance spherical ultrasonic waves (i.e. having a spherical wave front O1). The control system may be programmed such that the ultrasonic waves are transmitted at a rate of several thousand ultrasonic waves per second, for instance more than 10 000 unfocussed ultrasonic waves per second.

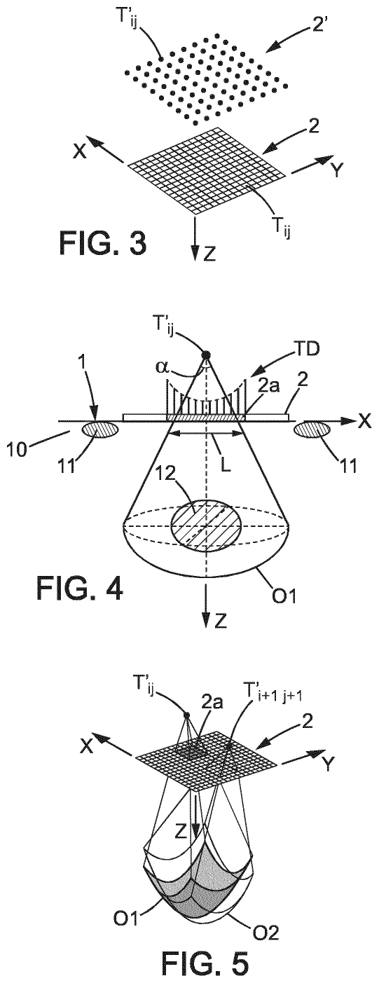

Spherical waves can be generated by a single transducer element (with low amplitude) or more advantageously with higher amplitude by a large part of the matrix array using one or more virtual point sources T′ᵢⱼ forming a virtual array 2′ placed behind of in front of the 2D array ultrasonic probe 2, as shown in FIGS. 3-4. The transmit delay TD applied by the control system to a transducer element e placed in position

$$( \begin{matrix}{x}_{e} \\ {y}_{e} \\ 0\end{matrix} )$$

associated to the virtual source v placed in position

$$( \begin{matrix}{x}_{v} \\ {y}_{v} \\ {z}_{v}\end{matrix} )$$

is:

TD=\sqrt{zᵥ²+(xₑ−xᵥ)²+(yₑ−yᵥ)²}/c

where c is the speed of sound.

For each virtual source T′ᵢⱼ used, it is possible for the control system to activate only a subset 2a of the 2D array ultrasonic probe 2, having a sub-aperture L which determines the aperture angle α of the divergent ultrasonic wave. The aperture angle α may be for instance of 90°. The imaged depth along axis Z may be about 12 to 15 cm.

It is possible to use only one virtual source and thus one ultrasonic wave for each 3D image of the heart, as will be explained later.

However, to enhance image resolution and contrast, it is useful to transmit the unfocused ultrasonic waves in series of successive unfocussed ultrasonic waves, the successive unfocused ultrasonic waves of each series having respectively different propagation directions: in that case, each 3D image is synthesized from the signals acquired from one of said series of successive unfocussed ultrasonic waves as will be explained later. The successive ultrasonic waves of each series may be obtained by varying the virtual source Tᵢⱼ from one wave to the other, thus varying the wave front O1, O2 etc., as shown in FIG. 5. Each series may include for instance 5 to 20 successive ultrasonic waves of different directions, for instance 10 to 20 successive ultrasonic waves of different directions.

In all cases, after each ultrasonic wave is transmitted, backscattered echoes are acquired by said 2D array ultrasonic probe (sampled for instance with a sampling rate of 12 MHz) and memorized. This raw data (also usually called RF data or radiofrequency data) is the used to generate a sequence of 3D images.

The duration of acquisition may be comprised between 10 ms and a few cardiac cycles, for instance at least one part of the cardiac cycle (for instance the diastole or systole, or one cardiac cycle) and less than 10 cardiac cycles (for instance less than 5 cardiac cycles). Such duration may be for instance comprised between 1 s and 10 s (for instance less than 5s). In a specific example, such duration is around 1.5s.

An electrocardiogram (ECG) may be co-recorded during the acquisition.

(b) Imaging:

After receiving the backscattered echoes, a parallel beamforming may be directly applied by the control system to reconstruct the 3D image from each single ultrasonic wave. Delay and sum beamforming can be used in the time domain or in the Fourier domain. In the time domain, the delays applied on the signal received by each transducer element e to reconstruct a voxel placed in

$$( \begin{matrix}x \\ y \\ z\end{matrix} )$$

is the sum of the forward propagation time from the virtual source v to the voxel and the backscattered propagation time to the transducer element e:

Delay=forward delay+Backscattered delay

Forward delay=\sqrt{(z−zᵥ)²+(x−xᵥ)²+(y−yᵥ)²}/c

Backscattered delay=\sqrt{z²+(x₆−x)²+(y₆−y)²}/c

In case the ultrasonic waves are transmitted by series of ultrasonic waves having respectively different propagation directions as explained above, each image can be obtained by the control system through known processes of synthetic imaging. Voxels are beamformed using delay-and-sum algorithms for each virtual source and subsequently coherently compounded to form a final, high quality 3D image. Details of such synthetic imaging can be found for instance in:
Montaldo, G., Tanter, M., Bercoff, J., Benech, N., Fink, M., 2009. Coherent plane-wave compounding for very high frame rate ultrasonography and transient elastography. IEEE Trans. Ultrason. Ferroelectr. Freq. Control 56, 489-506. doi:10.1109/TUFFC.2009.1067
Nikolov, S. I., 2001. Synthetic aperture tissue and flow ultrasound imaging. Orsted-DTU, Technical University of Denmark, Lyngby, Denmark.
Nikolov, S. I., Kortbek, J., Jensen, J. A., 2010. Practical applications of synthetic aperture imaging, in: 2010 IEEE Ultrasonics Symposium (IUS). Presented at the 2010 IEEE Ultrasonics Symposium (IUS), pp. 350-358. doi:10.1109/ULTSYM.2010.5935627
Lockwood, G. R., Talman, J. R., Brunke, S. S., 1998. Real-time 3-D ultrasound imaging using sparse synthetic aperture beamforming. IEEE Trans. Ultrason. Ferroelectr. Freq. Control 45, 980-988. doi:10.1109/58.710573
Papadacci, C., Pernot, M., Couade, M., Fink, M. & Tanter, M. High-contrast ultrafast imaging of the heart. IEEE transactions on ultrasonics, ferroelectrics, and frequency control 61, 288-301, doi:10.1109/tuffc.2014.6722614 (2014).

The framerate, i.e. the rate of 3D images in the animated sequence which is finally obtained, may be of several thousand 3D images per second, for instance 3000 to 5000 3D images per second.

(c) Blood and Tissue Velocity Computing:

Blood flow and tissue motion estimation may be performed by the control system using known methods.

For instance, the Kasai algorithm may be used to estimate motion in blood and in tissues with a half-wavelength spatial sampling (Kasai, C., Namekawa, K., Koyano, A., Omoto, R., 1985. Real-Time Two-Dimensional Blood Flow Imaging Using an Autocorrelation Technique. IEEE Trans. Sonics Ultrason. 32, 458-464. doi:10.1109/T-SU.1985.31615). Blood flow can be estimated by first applying a high-pass filter to the baseband data and then, for each individual voxel, Power Doppler may be obtained by integrating the power-spectral density, Pulsed Doppler may be obtained by computing the short-time Fourier transform, and Color Doppler maps may be obtained by estimating the first moment of the voxel-specific Pulsed-Doppler spectrogram. Power velocity integral maps can be obtained by computing the time integral of power times velocity in order to obtain images of a parameter related to flow rate. Advanced filtering such as Spatio-temporal filters based on singular value decomposition can also be used to better remove the clutter signal (Demené, C. et al. Spatiotemporal Clutter Filtering of Ultrafast Ultrasound Data Highly Increases Doppler and Ultrasound Sensitivity. IEEE transactions on medical imaging 34, 2271-2285, doi:10.1109/tmi.2015.2428634 (2015)).

In a specific example:
4D tissue velocity may be computed by performing 1D cross-correlation to obtain volumes of tissue volume-to-volume axial displacements. A butter-worth low-pass filtering with a 60 Hz cut-off frequency was then applied on the displacements. A myocardium 3D mask (specific to the tissues of the myocardium) may be applied to remove signal outside the muscle. To display 4D tissue velocities, Amira® software may be used. In each voxel, one tissue velocity curve may be derived.
4D Color Doppler may be computed by performing an SVD filtering to remove signal from the tissue and keep only the signal from the blood flow as it is done for instance in the above publication by Demené et al. 1D axial cross-correlation pixel-per-pixel on SVD-filtered voxels may be performed to obtain Color Doppler volumes. To display 4D Color Doppler, a cavity 3D mask (specific to the cavity receiving blood, e.g. the left ventricular cavity) may be applied to remove signal outside the cavity and the volume rendering may be performed using Amira® software.

The above mentioned 3D masks of the left ventricle cavity and myocardium may be computed as follows. The cavity may be segmented using Power Doppler flow integrated over the entire cardiac cycle on the 3D images. The myocardium may be segmented using integrated tissue velocity over the cardiac cycle and manual selection of the contour on two perpendicular 2D slices. An elliptic interpolation may be used to get the three-dimensional representation.

More generally, step (c) involves automatically computing 3D cartography of at least one parameter related to blood velocity and/or tissue velocity in said imaged volume, based on said sequence of 3D images. Said 3D cartography may consist of an animated sequence of 3D images of the computed parameter. The parameter may be blood and/or tissue velocity, or a component thereof.

(d) Locating of Points of Interest:

Depending on the quantification parameters which are sought, at least one point of interest having a predetermined property is automatically located by the control system in the sequence of 3D images.

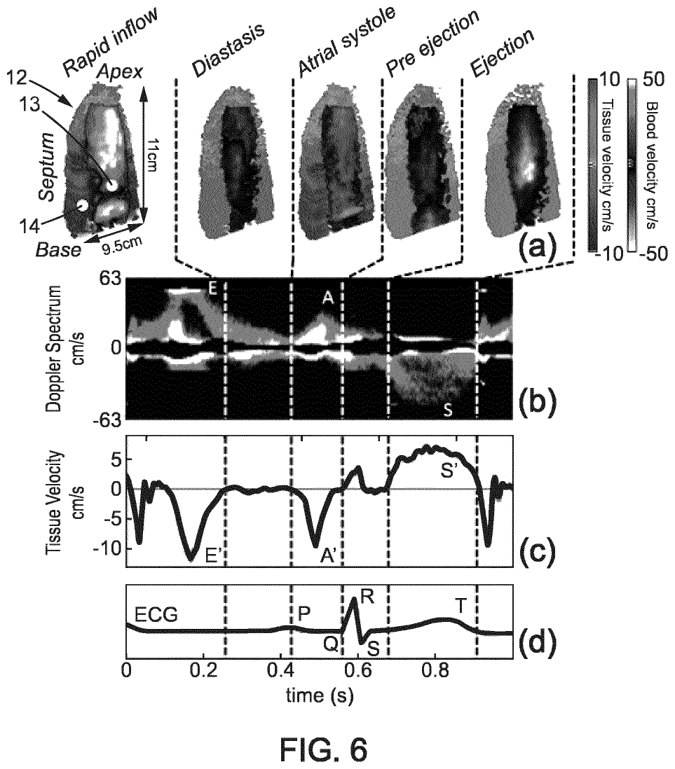

When the quantification parameter involves a peak blood velocity in a certain anatomic area, the control system may automatically locate said point of interest as a point of maximum blood velocity in said anatomic area and in at least part of the sequence of 3D images. For instance, when the early diastolic trans-mitral flow velocity E has to be computed, i.e. the peak blood velocity through the mitral valve during the E wave of the cardiac cycle (early diastolic wave), the control system (and more particularly computer 4) may automatically spot the point (FIG. 6a) of peak blood velocity inside the mitral valve. In a specific example, a Fourier transform over time may be performed at each voxel using a 60 sample sliding window to retrieve a spectrogram everywhere in the volume. Automatic dealiasing may be performed according to the above Demené et al. The location of point 13 may then be automatically detected by detecting the blood flow maximum.

When the quantification parameter involves tissue velocity at a certain anatomic position in the heart, the control system may automatically locate said anatomic position in the sequence of 3D images. For instance, when the early diastolic mitral annular velocity E′ has to be computed (i.e. the velocity of the mitral valve during the E wave of the cardiac cycle) the control system (and more particularly computer 4) may automatically spot a point 14 of the mitral valve (FIG. 6a). Such automatic location may be done according to an anatomic model of the heart memorized in computer 4, or by selecting a point in the tissues in correspondence with the above point 14 of maximum blood velocity.

When the quantification parameter involves a peak tissue velocity in a certain anatomic area, the control system may automatically locate said anatomic area in the sequence of 3D images and said point of interest as a point of maximum tissue velocity in said anatomic area in the sequence of 3D images. For instance, when the peak systolic annular velocity S′ of the left ventricle has to be computed, the system determines a point (not shown) of the tissues surrounding the ventricle having the maximum velocity in the image sequence myocardium.

(e) Quantification

The desired quantification parameter(s) can then be computed by the control system (and in particular by computer 4) based on the previously determined point(s) of interest, and based on the peak blood or tissue velocity of such point of interest.

Particularly useful examples of such quantification parameters are E, A, E′, A′, S, D, Vp, S′, E/A, E/E′, E/E′, E′/A′, S, S/D, Q, Qsystolic, Qdiastolic, DT, IVRT, PVAT, VTI, Gmean and Gmax wherein:
E is the early diastolic trans-mitral flow velocity as defined above;
E′ is the early diastolic mitral annular velocity as defined above (computed at the instant of peak blood velocity corresponding to E);
A is late diastolic trans-mitral flow velocity;
A′ is the late diastolic mitral annular velocity (computed at the instant of peak blood velocity corresponding to A);
S is the peak pulmonary venous systolic velocity;
D is the peak pulmonary venous early diastolic velocity;
Vp is the velocity of flow progression;
Q is the flow rate or cardiac output;
Qsystolic is the total output transaortic flow rate;
Qdiastolic is the total input transmitral flow rate;
DT is the e-wave deceleration time;
PVAT is the pulmonary acceleration time;
IVRT is the length of the isovolumetric relaxation time;
Gmean and Gmax are the mean and maximum transvalvular pressure gradients;
VTI is velocity time integral;
S′ is the peak systolic annular velocity as defined above.

It should be noted that:
at step (d), said at least one point of interest is automatically located based solely on said 3D cartography and its temporal profile;
and at step (e), said at least one velocity is automatically determined at said at least one point of interest based solely on said 3D cartography and its temporal profile.

More generally, in the present disclosure the transvalvular blood flows can be localized using only the spatial and temporal velocity information without any additional anatomic information. Temporal profiles of the flow velocity (or Spectral Doppler profile) are indeed a strong characteristic of the valve location and are very specific to the type of valve:

Transaortic blood flow is characterized by a strong outflow during the entire systole, followed by no flow (or lower flow in the reverse direction in case of aortic regurgitation) during the diastole. The transaortic flow can then be localized precisely by determining the spatial peak of the outflow blood velocity.

In contrast, transmitral blood flow is characterized by no or little flow in systole and two inflow peaks in early and late diastole. The transmitral flow can then be localized precisely by finding the spatial peak of the inflow blood velocity.

This is made possible in particular because the temporal profiles are obtained simultaneously at every voxel of the image.

The point and interest and the velocity at this point of interest are thus determined without need of anatomical image, in particular without need of a B-mode anatomical image, thanks to the fact that the present method involves determining the 3D cartography of velocity in the whole imaged volume. Thus, the whole method of the present disclosure need no B-mode imaging, and more generally no anatomical imaging, which enables quicker results of the present method.

### EXAMPLE

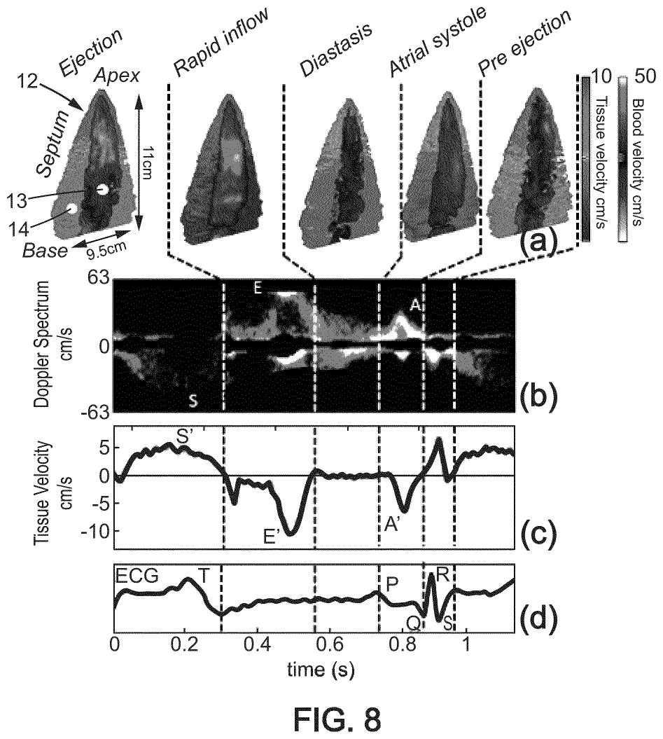

The left ventricle of a healthy human volunteer and a young patient with a hypertrophic cardiomyopathy were imaged (respectively FIGS. 6 and 8) and the index E/E′ was automatically computed for both cases.

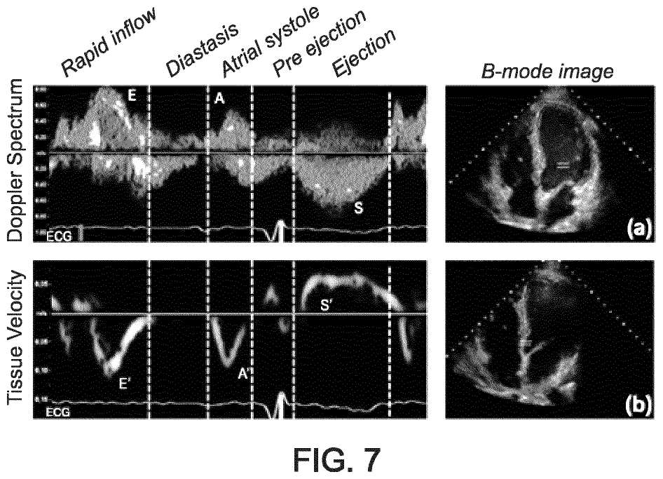

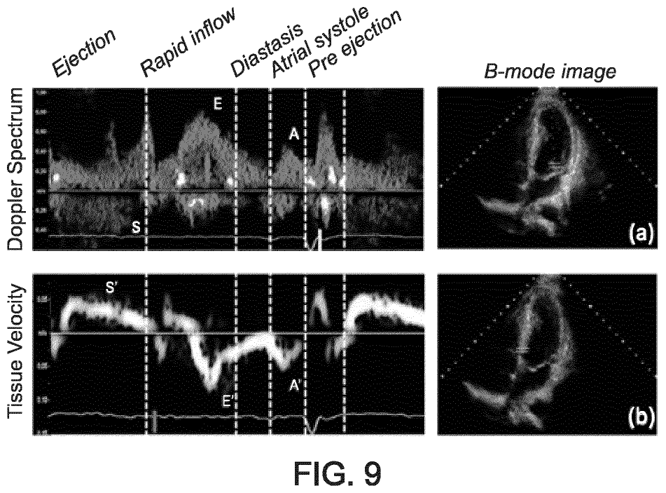

The healthy human volunteer and the young patient were then scanned by a cardiologist using a classical clinical ultrasound system on the apical 4-chambers view (FIGS. 7 and 9). Doppler spectrum and tissue velocity were assessed using pulsed Doppler and tissue Doppler modes and the index E/E′ was automatically computed for both cases. These manual measures confirmed the accuracy of the automatic measurement, while the automatic measurement is much quicker (it is done in one heartbeat) and does not require specific training for the operator.

1. Method for 4D imaging of a heart of a living being, said method including at least the following steps:
(a) an acquisition step wherein unfocused ultrasonic waves are transmitted in the heart by a 2D array ultrasonic probe and raw data from backscattered ultrasonic waves are acquired by said 2D array ultrasonic probe;
(b) an imaging step wherein a sequence of 3D images is generated from said raw data, said sequence of 3D images forming an animation showing movements of an imaged volume including at least part of the heart;
(c) a velocity computing step wherein 3D cartography of at least one parameter related to blood velocity and tissue velocity are automatically computed in said imaged volume and time, based on said sequence of 3D images;
(d) a locating step wherein at least one point of interest having a predetermined property is automatically located in said sequence of 3D images;
(e) a quantification step wherein at least one velocity chosen between blood velocity and tissue velocity is automatically determined at said at least one point of interest and a predetermined quantification parameter is automatically computed, involving said at least one velocity,
wherein the quantification parameter involves:
either a peak blood velocity in a certain anatomic area and said locating step (d) includes automatically locating said point of interest as a point of maximum blood velocity in said anatomic area and in at least part of the sequence of 3D images;
or a peak tissue velocity in a certain anatomic area and said locating step (d) includes automatically locating said anatomic area in the sequence of 3D images and automatically locating said point of interest as a point of maximum tissue velocity in said anatomic area in the sequence of 3D images.

2. Method according to claim 1, wherein the quantification parameter involves a temporal variation of the parameter related to the velocity in a certain anatomic area and said locating step (d) includes automatically locating said point of interest as a point of maximum blood velocity in said anatomic area and in at least part of the sequence of 3D images.

3. Method according to claim 1, wherein the quantification parameter involves a time integral of the parameter related to the velocity in a certain anatomic area and said locating step (d) includes automatically locating said point of interest as a point of maximum blood velocity in said anatomic area and in at least part of the sequence of 3D images.

4. Method according to claim 1, wherein the quantification parameter involves a space integral of the parameter related to the velocity in a certain anatomic area and said locating step (d) includes automatically locating said point of interest as a point of maximum blood velocity in said anatomic area and in at least part of the sequence of 3D images.

5. Method according to claim 1, wherein the quantification parameter determined at said quantification step is chosen from E, A, E′, A′, S, D, Vp, S′, E/A, E/E′, E/E′, E′/A′, S, S/D, VTI, Gmean and Gmax wherein:
E is the early diastolic trans-mitral flow velocity;
E′ is the early diastolic mitral annular velocity;
A is late diastolic trans-mitral flow velocity;
A′ is the late diastolic mitral annular velocity;
S is the peak pulmonary venous systolic velocity;
D is the peak pulmonary venous early diastolic velocity;
Vp is the velocity of flow progression;
Gmean and Gmax are the mean and maximum transvalvular pressure gradients;
VTI is velocity time integral;
S′ is the peak systolic annular velocity.

6. Method according to claim 1, wherein during said acquisition step (a), said unfocused ultrasonic waves are transmitted in several series of successive unfocused ultrasonic waves, the successive unfocused ultrasonic waves of each series having respectively different propagation directions, and said imaging step (b) includes synthesizing a 3D image by ultrasound synthetic imaging from the respective raw data corresponding to said successive unfocussed ultrasonic waves of each series.

7. Method according to claim 1, wherein:
at step (d), said at least one point of interest is automatically located based solely on said 3D cartography and its temporal profile;
and at step (e), said at least one velocity is automatically determined at said at least one point of interest based solely on said 3D cartography and its temporal profile.

8. Apparatus for 4D imaging of a heart of a living being, said apparatus including at least a 2D array ultrasonic probe and a control system configured to:
(a) transmit unfocussed ultrasonic waves in the heart through said 2D array ultrasonic probe and acquire raw data from backscattered ultrasonic waves through said 2D array ultrasonic probe,
(b) generate a sequence of 3D images from said raw data, said sequence of 3D images forming an animation showing movements of an imaged volume including at least part of the heart,
(c) automatically compute 3D cartography of at least one parameter related to blood velocity and tissue velocity in said imaged volume, based on said sequence of 3D images,
(d) automatically locate at least one point of interest having a predetermined property in said sequence of 3D images,
(e) automatically determine at least one velocity chosen between blood velocity and tissue velocity at said at least one point of interest and automatically compute a predetermined quantification parameter involving said at least one velocity, wherein the quantification parameter involves:
either a peak blood velocity in a certain anatomic area and said control system configured to automatically locate said point of interest as a point of maximum blood velocity in said anatomic area and in at least part of the sequence of 3D images;
or a peak tissue velocity in a certain anatomic area and said control system configured to automatically locate said anatomic area in the sequence of 3D images and automatically locating said point of interest as a point of maximum tissue velocity in said anatomic area in the sequence of 3D images.

9. Apparatus according to claim 8, wherein the quantification parameter is chosen from E, A, E′, A′, S, D, Vp, S′, E/A, E/E′, E/E′, E′/A′, S, S/D, VTI, Gmean and Gmax wherein:
E is the early diastolic trans-mitral flow velocity;
E′ is the early diastolic mitral annular velocity;
A is late diastolic trans-mitral flow velocity;
A′ is the late diastolic mitral annular velocity;
S is the peak pulmonary venous systolic velocity;
D is the peak pulmonary venous early diastolic velocity;
Vp is the velocity of flow progression;
Gmean and Gmax are the mean and maximum transvalvular pressure gradients;
VTI is velocity time integral;
S′ is the peak systolic annular velocity.

10. Apparatus according to claim 8, wherein said control system is configured to transmit said unfocussed ultrasonic waves in several series of successive unfocussed ultrasonic waves, the successive unfocussed ultrasonic waves of each series having respectively different propagation directions, and said control system is configured to synthesize a 3D image by ultrasound synthetic imaging from the respective raw data corresponding to said successive unfocussed ultrasonic waves of each series.

11. Apparatus according to claim 8, wherein said control system is configured to transmit said unfocussed ultrasonic waves as divergent ultrasonic waves.

12. Apparatus according to claim 8, wherein said control system is configured to transmit said unfocussed ultrasound waves for a duration of at least part of a cardiac cycle and less than 10 cardiac cycles.

13. Apparatus according to claim 8, wherein said control system is configured to transmit said unfocussed ultrasound waves for a duration comprised between 1s and 10s.

14. Apparatus according to claim 8, wherein said control system is configured to:
(d) automatically locate said at least one point of interest based solely on said 3D cartography and its temporal profile;
(e) automatically determine said at least one velocity at said at least one point of interest based solely on said 3D cartography and its temporal profile.
